(19) United States
(12) Patent Application Publication
(10) Pub. No.: US 2009/0210000 A1
(43) Pub. Date: Aug. 20, 2009

(54) PERCUTANEOUS PEDICLE PLUG AND METHOD OF USE

(76) Inventors: Humbert G. Sullivan, La Crosse, WI (US); Aaron C. Mueller, Rochester, MN (US)

Correspondence Address: Intellectual Property Dept.;Dewitt Ross & Stevens SC, 2 East Mifflin Street, Suite 600, Madison, WI, 53703-2865, US

(21) Appl. No.: 12/371,813

(22) Filed: Feb. 16, 2009

Related U.S. Application Data
(60) Provisional application No. 61/065,989, filed on Feb. 15, 2008.

(51) Int. Cl. A61B 17/08

(52) U.S. Cl. 606/213

(57) ABSTRACT

A percutaneous pedicle plug for use insertion into a screw opening in a vertebral body is described. The percutaneous pedicle plug includes a first end, a tapered second or tail end 44, a threaded body 46 to allow for threaded insertion of the plug into the screw opening of the vertebral body, and a longitudinal central channel extending from the first end to the second end.

### REFERENCE TO RELATED APPLICATION

The application claims priority to U.S. Provisional Application entitled “PERCUTANEOUS PEDICLE PLUG,” Ser. No. 61/065,989, filed Feb. 15, 2008, which is incorporated herein by reference in its entirety.

### FIELD OF THE INVENTION

The present invention is directed to the field of spinal surgery and specifically to facilitating the staging of complex pedicle screw-based spinal procedures.

### DESCRIPTION OF THE PRIOR ART

Staging complex pedicle screw-based spinal procedures may improve the outcome of patients undergoing this type of surgery. Staging complex pedicle screw-based spinal procedures may be facilitated by an initial percutaneous stage employing pedicle screw implants with pop-on-heads, such as Click' X screws (SYNTHES USA, LLC, West Chester, Pa.). The use of pedicle screws with pop-on heads at the initial percutaneous stage allows for small skin incisions and minimizes tissue trauma during insertion. The absence of heads also facilitates dissection around the screws at the second stage of open surgery. The pop-on-heads are applied later, after dissection has been completed, at open surgery, which is the final stage.

However, there are certain issues with the use of these systems. For example, with Click' X screws, the final bond between the head and the shaft of Click' X screws may not be strong enough to withstand loading in multiple planes as demanded by the above noted types of surgery.

However, surgeons may be slow to accept the cannulated version of these new screws for these critical, new percutaneous applications—continuing to prefer screws that are monoaxial and do not have pop-on heads. Other surgeons may want the advantages of percutaneous pre-placed markers and tracts for pedicle screws, but may, in addition, want to place all screws at the time of the open procedure. This would allow a wide choice of screws while still preserving the advantages of having pre-placed markers and tracts for any pedicle screw or combination of screws which one may wish to use.

Currently, the only way to address this issue and retain the advantages of small incision and minimal tissue trauma afforded by the headless screws is to percutaneously pre-place a guide wire (known to the art as a K-wire) K-wire fragments at the levels where screws other than Click' X screws are desired.

Reference is made to Ringstrom et al., the entirety of which is incorporated herein by reference, which establishes the feasibility, safety, and efficacy of using either percutaneously placed headless Click' X pedicle screws or percutaneously placed K-wire fragments to stage complex pedicle screw base spinal surgery (Ringstrom, M. J. et al., J Neurosurgery Spine 7:521-532; November, 2007). Ringstrom et al. is the first report the use of percutaneously placed headless cannulated screws or percutaneously placed K-wire fragments in a staging algorithm. This staging algorithm is referred to as the Pere-To-Delayed, Open Staging process (PDO-Staging process). The algorithm consists of:
1. Stage I: The percutaneous placement of cannulated, headless pedicle screws or K-wire fragments. Reference is made to FIG. 1 to illustrate percutaneous K-wire placement.
2. A delay of 2 to 6 weeks or longer, if desired, between stages.
3. Stage II: Reference is made to FIG. 2 to illustrate the Stage II percutaneous placement of a cannulated, headless pedicle screw which precedes the primary surgical procedure for attachment of heads and rods to the pre-placed screws. If a K-wire fragment is placed in Stage I, it will be removed in Stage II and replaced by a pedicle screw at Stage II. Reference is made to FIG. 2 to illustrate the process of replacing the K-wire fragment by a pedicle screw at Stage II. Note that this part of the procedure is open surgery. The skin line present in this figure is for reference only.
   According to this algorithm, at Stage I (FIG. 1), the locations and trajectories of the pedicle screws to be employed in a patient are marked by K-wire fragments. Referencing FIG. 1, there is generally a seven step process for the placement of a K-wire fragment. The skin line 8 is shown for reference. In Steps 1 and 2, a channel or opening 10 is created in a vertebral body 12 by means of a Jam-Sheady needle 14, known to the art, or the like. The Jam-Sheady needle 14 contains an internal stylet 16 which, when removed from the Jam-Sheady needle 14, in Step 3, leaves a channel 17 within the Jam-Sheady needle 14. In Steps 4 and 5, a K-wire fragment 18a is placed within the channel of the Jam-Sheady needle 14. The K-wire fragment 18a is created by cutting a standard K-wire 18 to a desired length by a cutting device 20. In Step 6, the fragment 18a of the K-wire 18 is then inserted into the vertebral body 12 by means of a blunt pusher rod 22. The Jam-Sheady needle 14 and the blunt pusher rod 22 are then removed in Step 7 leaving the K-wire fragment 18a in place within the channel 10 in the vertebral body 12.

Stage II, where all of the objectives of surgery are accomplished, follows the percutaneous stage after about two weeks (there are exceptions such as multiple trauma cases or more complex cases where Stage I is broken into multiple sub stages). As illustrated in FIG. 2, there are 7 steps for screw placement in Stage II when a K-wire fragment 18a has been placed in Stage I of the PDO-Staging process. Stage II is open surgery so the skin and intervening tissues have been opened down to the level of the spine. With respect to the figures, the skin line 8 is shown for reference only. In Steps 1 and 2, the K-wire fragment 18a is located and removed from the channel 10 of the vertebral body 12 by means known in the art. In Step 3, the K-wire fragment 18a is replaced with a longer K-wire 18b. Use of the longer K-wire 18b is necessary to secure the K-wire during subsequent steps. Typically, this process is x-rayed to ensure the K-wire 18b is not advanced outside of the vertebral body 12. In Step 4, a tap or pedicle probe 24 is placed over the K-wire 18b and is threaded into the vertebral body 12 to generate a screw opening 25 according to the size of a pedicle screw one plans to place within the vertebral body 12. In Step 5, the pedicle probe 24 is removed along with the K-wire 18b. In Steps 6 and 7, the pedicle screw 26 is then rotatably placed within the screw opening 25 of vertebral body 12.

For elective cases where the anesthesia time may exceed 6 hours or may exceed the tolerance of a fragile patient, the time delay between Stage I and Stage II is an essential feature of the PDO-Staging process. Sufficient time must be allowed between Stage I and Stage II to avoid additive effects of anesthesia between the two stages, which would increase the complication rate. In very fragile patients, Stage I may be accomplished in multiple sittings depending on the number of screws to be placed and how fragile the patient may be medically. In some very fragile children with scoliosis secondary to neuromuscular disease, more than 20 screws may be required. In such patients it may be quite advantageous to split Stage I into multiple small sub-stages over a more protracted period of time.

In the most general sense, Stage I refers to any percutaneous procedure or combination thereof to facilitate pedicle screw placement and, thereby, shorten the time of anesthesia during Stage II. The main advantage in Ringstrom et al. is that the percutaneous pedicle instrumentation in Stage I performed about two weeks prior to the open surgery of Stage II reduces the overall duration of the complex open surgery required for setting pedicle screws. This significantly reduces the complication rate of the surgery.

A disadvantage to this process is that, at the time of open surgery, each K-wire fragment must be removed, a longer K-wire placed, and the channel or opening must be threaded to the appropriate size for the screw to be used. These steps still require a substantial amount of time of open surgery.

Further, the time to perform each of these steps is multiplied by the number of screws, which in some cases may be 10 to 20 screws. This extra time is, therefore, added to the exponential end of the complications vs. rate curve for the open surgical procedure resulting in an undesirable, potentially large increase in the complication rate in such cases.

Further still, fluoroscopy must be used in the surgical suite to insure that the tap does not drive the K-wire into the abdominal or thoracic cavities with a potentially life threatening injury to vital organs or vasculature. This requirement for fluoroscopy adds anesthesia time and increases radiation exposure for the patient and staff.

In addition, the actual position of the screw, which follows the K-wire, cannot be known before surgery. One surgeon has opted not to replace the K-wire fragment with a long K-wire before tapping which has resulted in a K-wire being inadvertently driven through the vertebral body into the abdominal cavity.

Another potential issue with K-wire fragments is the possibility of migration of the K-wire fragment internally, resulting in injury to vascular structures or vital organs. At the time of open surgery, the sharp protruding end of the K-wire fragment also adds a small risk of penetration of the surgeon's glove, thus introducing an infection risk. The extra surgical steps and risk complicate the staging algorithm when K-wire fragments are used. This may discourage surgeons who do not want to use cannulated pre-placed, headless screws, from trying this process. At a more elementary level, surgeons may simply be hesitant to switch to an unfamiliar pedicle screw system.

### SUMMARY OF THE INVENTION

The present invention is directed to a percutaneous pedicle plug (“PERC-PLUG” device). The PERC-PLUG device is an elegant way to allow surgeons to take advantage of the PDO-Staging process and still employ any screw of their choice. The percutaneous pedicle plug for insertion into a screw opening in a vertebral body comprises a first end; a tapered second or tail end; a threaded body to allow for threaded insertion of the plug into the screw opening of the vertebral body; and a longitudinal central channel extending from the first end to the second end. The percutaneous pedicle plug may also include an extension extending from the threaded body to the second end.

The present invention is also directed to a method of inserting a percutaneous pedicle plug within a vertebral body, comprising creating a channel opening in the vertebral body, forming threads within the channel opening of the vertebral body and inserting the percutaneous pedicle plug within the threads in the channel of the vertebral body.

The present invention is further directed to a method of inserting a percutaneous pedicle plug within a vertebral body, comprising forming a channel opening in the vertebral body by means of a Jam-Sheady needle wherein the Jam-Sheady needle includes a needle channel extending therethrough, inserting a guide wire within the needle channel of the Jam-Sheady needle and the channel of the vertebral body, removing the Jam Sheady needle, inserting a threaded probe into the vertebral body channel wherein the threaded probe includes a channel for the guide wire, forming threads within the channel opening of the vertebral body by means of the threaded probe, removing the threaded probe and the guide wire from the vertebral body; and inserting the percutaneous pedicle plug within the threads in the channel of the vertebral body.

The present invention is also directed to a method of installing a percutaneous screw in a vertebral body comprising forming a channel opening in the vertebral body, forming threads within the channel opening of the vertebral body by means of the threaded probe, removing the threaded probe from the vertebral body, inserting a percutaneous pedicle plug within the threads in the channel of the vertebral body, wherein the percutaneous pedicle plug includes a first end, a tapered second or tail end, and a threaded body to allow for threaded insertion of the percutaneous pedicle plug into the channel of the vertebral body, a longitudinal central channel extending from the first end to the second end, and, after a designated period of time removing the percutaneous pedicle plug from the vertebral body, and finally installing the percutaneous screw within the channel in the vertebral body.

Advantageously, the PERC-PLUG device will significantly complement and encourage the use of pedicle screw-based products currently in the market; thereby reducing the need for other less desirable techniques such as sub-laminar wiring, sub-laminar hooks, facet hooks, pedicle hooks or transverse process hooks. These alternative methods of spinal fixation tend to be used in situations where it would be very difficult and risky to attempt pedicle screw placement at open surgery. Other advantages include the following:

Accuracy of Screw Placement: The accuracy of screw placement by PERC-PLUG device of the present invention may be superior to the usual methods employed at open surgery. The method of placement of the PERC-PLUG device also lends itself to accurate placement in difficult situations such as where the vertebrae have rotated in the coronal plane. Also, prior to the open surgery one can identify any misplaced placed screws, which is very important in these complex cases.

Cost Savings: The high degree of accuracy obviates the need for very expensive and time consuming methods to improve accuracy such as the use of intra-operative computed tomography (CT) or guidance systems. In addition, reduction of complication rate and reduction of operating room time will significantly reduce cost.

Time Savings: Furthermore, the time savings in these long cases may be magnified by the exponential nature of the relationship between rate of complications and time. When using the PERC-PLUG device, each screw can be placed in a matter of seconds as compared to several minutes for a K-wire fragment site. Considering the simplicity and the reduction in the number of steps, placing any desired pedicle screw during Stage II when a PERC-PLUG device was placed during Stage I is very time efficient compared to the K-wire fragment technique.

Reduction in the Number of Required Steps: The present invention dramatically alters the way complex, pedicle screw-based spinal surgery is performed. The PERC-PLUG device of the present invention is an alternative to the use of K-wire fragments in the PDO-Staging process. However, by placing the PERC-PLUG device during Stage I, there are only 3 steps for placing the desired pedicle screw:
1. Find the PERC-PLUG device.
2. Remove the PERC-PLUG device.
3. Place the pedicle screw.

No Artifacts: The PERC-PLUG device does not produce artifacts on magnetic resonance (MR) or CT scans. In advance of the open surgery one can know very precisely what the track of each screw will be and whether or not there have been any violations of the pedicle cortex.

Avoids Risk of Migration, Inadvertent K-wire Advancement, or Glove Puncture: The PERC-PLUG device avoids the risk of migration, the risks of inadvertent advancing of the K-wire during placement of the fragment at Stage I or during tapping at Stage II, and the risk of puncture of the surgeon's glove when finding the K-wire during Stage II.

No Intra-operative X-ray Required: With the PERC-PLUG device, there is no intra-operative x-ray required for location of incision or for screw placement. The small incisions for the placement of the PERC-PLUG devices mark the location of the skin incision at open surgery and screws are simply passed down the preexisting holes in the pedicles.

Allow CT Imaging of the Future Screw Location: An important benefit of the use of PERC-PLUG devices is that they allow CT imaging of the future screw location without any metal artifact. This allows for precise information about future screw location relative to the boundaries of the pedicle. In other designs, it may be very difficult to know if the walls of the pedicle have been violated by a metallic screw without special algorithms or manipulation of CT data to account for metal artifacts on CT. Utilizing the PERC-PLUG devices, the surgeon can enter the operating room for Stage II of the procedure with precise knowledge of the future location of all of the screws, that is, where PERC-PLUG devices have been used.

Flexibility of the PERC-PLUG Device: Another important point in favor of the use of PERC-PLUG devices is that with their use the surgeon may have all the advantages of PERC-PLUG device and at the same time be able to place any desired screw at any point in the construct. This is truly the best of all possible worlds.

The objects and advantages of the invention will appear more fully from the following detailed description of the preferred embodiment of the invention made in conjunction with the accompanying drawings.

### BRIEF DESCRIPTION OF THE DRAWINGS

FIG. 1 is a prior art schematic view illustrating the seven steps of percutaneous placement of cannulated, headless pedicle screws or K-wire fragments at Stage I in the prior art.

FIG. 2 is a prior art schematic view illustrating the seven steps of Stage II pedicle screw placement after the K-wire placement in Stage I.

FIGS. 3a-c are schematic views illustrating a first (Type 1) version of the PERC-PLUG device of the present invention in association with a vertebra and in association with a K-wire.

FIGS. 4a-c are schematic views illustrating a second (Type 2) version of the PERC-PLUG device of the present invention in association with a vertebra and in association with a K-wire.

FIG. 5 is a schematic view illustrating the ten steps of PERC-PLUG device placement during the Stage I procedure.

FIG. 6 is a schematic view illustrating the three steps of Stage II screw placement after a PERC-PLUG device at Stage I.

### DETAILED DESCRIPTION OF THE INVENTION

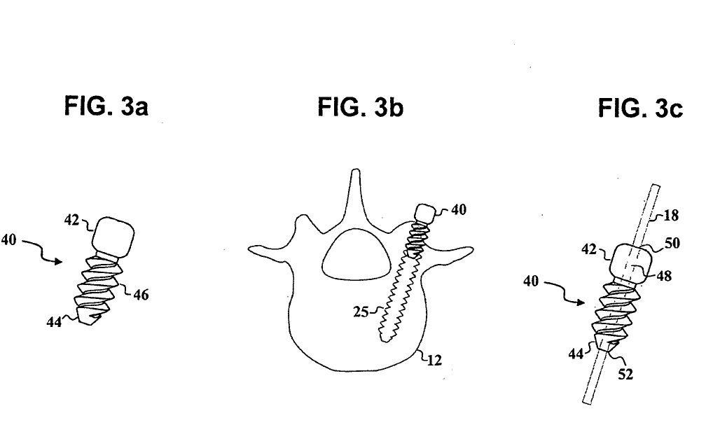

Reference is now made to FIGS. 3a-c for a description of one embodiment (termed Type 1) of the PERC-PLUG device 40 of the present invention. As illustrated in FIG. 3a, the PERC-PLUG device 40 is a relatively short device having a smooth first or head end 42, a tapered second or tail end 44, and a threaded body 46 to allow for threadable insertion of the PERC-PLUG device 40 into the screw opening 25 of the vertebral body 12 as illustrated in FIG. 3b. Referring to FIG. 3c, the PERC-PLUG device 40 also includes a longitudinal channel 48 extending from a first opening 50 in the head end 42 to a second opening 52 in the tail end 44. The longitudinal channel 48 is dimensioned and configured to receive a K-wire 18 therethrough. The PERC-PLUG device 40 resembles a pedicle screw 26 which has been amputated after the first several turns of its thread.

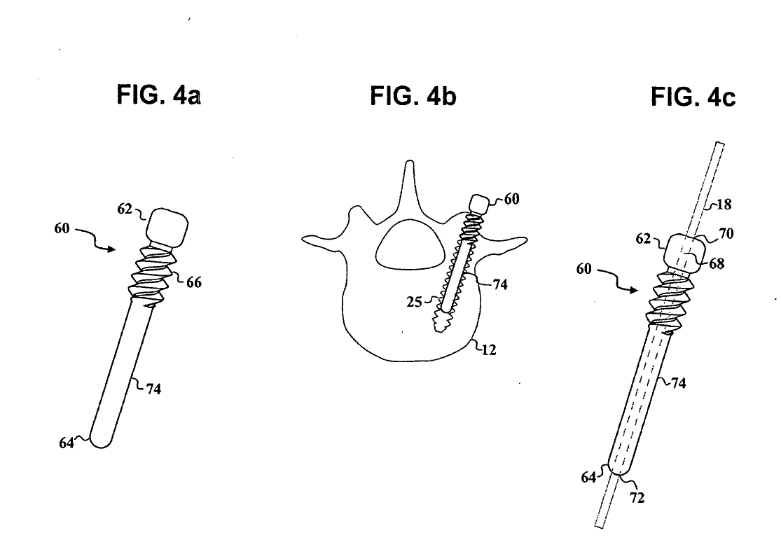

Referring now to FIGS. 4a-c, there is illustrated an alternative embodiment of a PERC-PLUG device 60, identified as a Type-2 PERC-PLUG device. As shown in FIG. 4a, the Type-2 PERC-PLUG device 60 also resembles a pedicle screw 26 at the head end. Further, the Type-2 PERC-PLUG device 60 resembles the Type-1 PERC-PLUG device 40 in that the Type-2 PERC-PLUG device 60 also has a smooth first or head end 62, a tapered second or tail end 64, and preferably a threaded body 66 to allow for threadable insertion of the PERC-PLUG device 60 into the screw opening 25 of the vertebral body 12 as illustrated in FIG. 4b, and, referring to FIG. 4c, a longitudinal channel 68 extending from a first opening 70 in the head end 62 to a second opening 72 in the tail end 64 for receiving a K-wire 18. However, the Type-2 PERC-PLUG device 60 is distinguished from the Type-1 PERC-PLUG device 40 by the addition of a smooth threadless extension 74 below the threaded body 66. The head end 62 (or 42 in FIG. 3c), designed to protrude from the vertebral body 12, is elongated to allow it to be located easily at the time of open surgery but also designed so that it will be easy to work around in the process of surgical dissection.

In the remaining discussion, the reference of the PERC-PLUG device 40 or the PERC-PLUG device 60 can and will be used interchangeably unless specific reference is made to one or the other. In the absence of specific direction, reference to the PERC-PLUG device 40 is intended to also refer to the PERC-PLUG device 60, and vice versa.

Preferably, the material comprising the PERC-PLUG device 40 (or 60) is biocompatible, radio-opaque or capable of containing a radio-opaque marker, non-artifact producing, and relatively inexpensive. Non-limiting examples of such materials include biocompatible plastics or polycarbonates marked for radiological identification. An exemplary, but non-limiting, material for constructing the PERC-PLUG device 40 (or 60) is poly-ethyl-ether-ketone (PEEK).

It is within the scope of the present invention to mix the material with barium in the curing stage, or a titanium bead could be located at a specific point within the device. The barium provides a homogeneous medium which is radio-opaque and non artifact producing. The titanium bead would allow radiological location of an otherwise radio-lucent device with minimal artifact on CT.

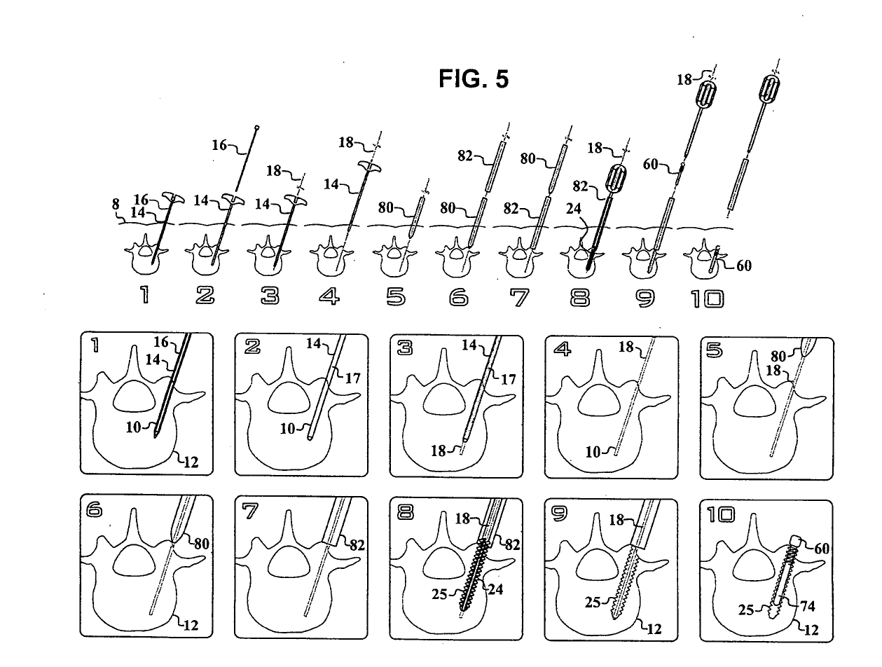

Referring to FIG. 5, the PERC-PLUG device 40 (or 60) is inserted during Stage I of the PDO-Staging process. The skin line 8 is shown for reference. In Step 1, a channel or opening 10 is created in a vertebral body 12 by means of a Jam-Sheady needle 14. The Jam-Sheady needle 14 contains an internal stylet 16 which, when removed from the Jam-Sheady needle 14 in Step 2 leaves a channel 17 within the Jam-Sheady needle 14. In Step 3, a long K-wire 18 is inserted within the channel 17 of the needle 14. The needle 14 is then removed in Step 4, leaving the K-wire 18 within the channel 10. In Step 5, a small hollow dilator 80 is slid over the long K-wire 18. In Step 6, a hollow working channel 82 is inserted over the tissue dilator 80. In Step 7, the small dilator 80 is removed leaving the long K-wire inside the hollow working channel 82. In Step 8, a tap or pedicle probe 24 (which is the size of the pedicle screw to be placed later) is placed over the K-wire 18 through the hollow working channel 82. This tap or pedicle probe 24 is then used to create the screw opening 25 in the vertebral body 12. This screw opening 25 will later receive the pedicle screw 26. In Step 9, the pedicle probe or tap 24 is removed and the PERC-PLUG device 40 or 60 is inserted percutaneously over the K-wire 18 into the vertebral body 12. In Step 10 all of the instruments are removed, leaving the PERC-PLUG device 40 or 60 in place within the vertebral body 12. The PERC-PLUG device 40 or 60 occupies only the first few turns in the screw opening 25 of the vertebral body 12. Initially, the PERC-PLUG device 40 or 60 provides hemostasis for the opening in the screw opening 25. The screw opening 25, which is anterior to the PERC-PLUG 40 or 60, is ready to receive any type of pedicle screw 26 a surgeon might want to place at open surgery.

The Type-2 PERC-PLUG device 60 with the threadless extension 74, as illustrated in Step 10 of FIG. 5, is designed to be used in situations where months or even a year may intervene between Stage I and Stage II. The threadless extension 74 of the PERC-PLUG device 60, when inserted within the vertebral body 12, prevents the screw opening 25 from filling in with bone over this long time interval between stages.

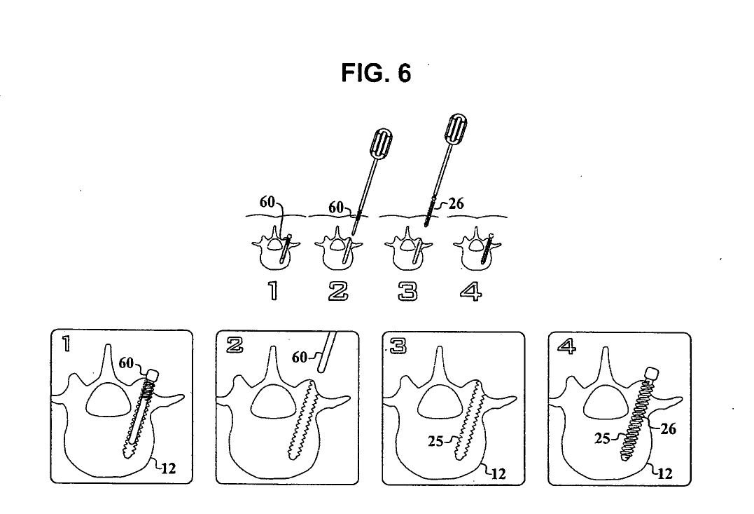

As illustrated in FIG. 6, Stage II of the present method comprises 3 steps for placing the desired pedicle screw 26 in the vertebral body 12. This occurs after a PERC-PLUG device 40 (or 60) has been placed within the vertebral body 12 during Stage I (as shown in FIG. 5). In Step 1 of Stage II, the PERC-PLUG device 60 is located. In Step 2, the PERC-PLUG device 60 is removed. Finally, in Step 3 (see panels 3 and 4), the pedicle screw 26 is placed in the screw opening 25 of the vertebral body 12.

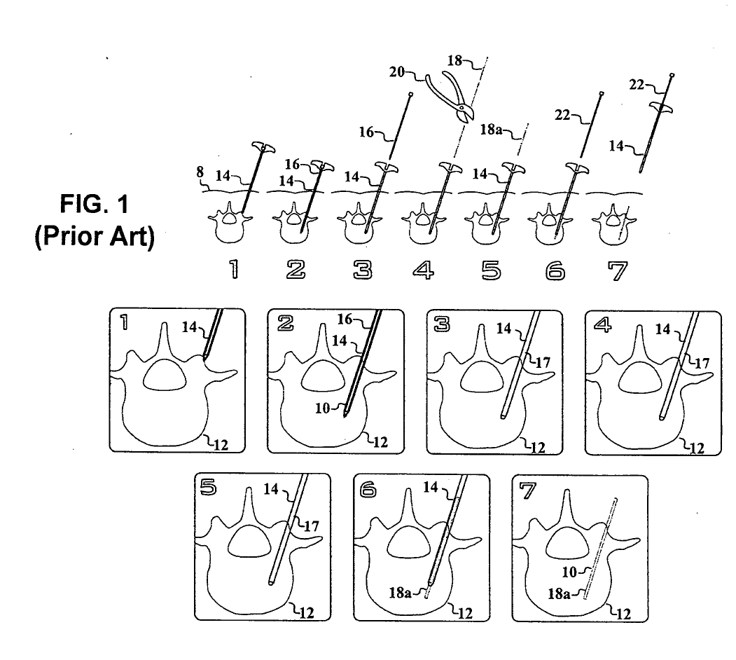

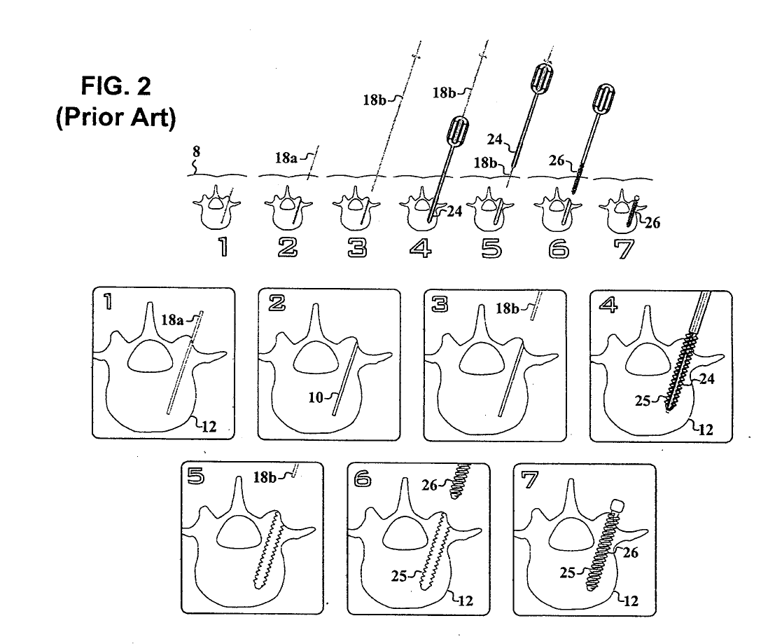

Therefore, in comparing Stage I and II of the prior art (FIGS. 1 and 2) with Stage I and II of the present invention (FIGS. 5 and 6), it is apparent that with the present invention more of the procedures involved with pedicle screw 26 placement can be performed percutaneously during Stage I. This greatly reduces the amount of steps required in Stage II, simplifies Stage II, and reduces the amount of time in open surgery.

It is understood that the invention is not confined to the particular construction and arrangement of parts herein illustrated and described, but embraces such modified forms thereof as come within the scope of the following claims.

What is claimed is:

1. A percutaneous pedicle plug for use insertion into a channel in a vertebral body, comprising:
a. a first end;
b. a tapered second or tail end;
c. a threaded body to allow for threaded insertion of the plug into the channel of the vertebral body; and
d. a longitudinal central channel extending from the first end to the second end.

2. The percutaneous pedicle plug of claim 1 wherein the threaded body comprises an extension extending from the threaded body to the second end.

3. The percutaneous pedicle plug of claim 2 wherein the extension includes a smooth exterior surface.

4. The percutaneous pedicle plug of claim 1 wherein the plug comprises materials selected from the group consisting of biocompatible plastics and polycarbonates.

5. The percutaneous pedicle plug of claim 1 wherein the plug is radio-opaque.

6. The percutaneous pedicle plug of claim 1 wherein the plug is capable of containing a radio-opaque marker.

7. The percutaneous pedicle plug of claim 1 wherein the plug comprises poly-ethyl-ether-ketone.

8. The percutaneous pedicle plug of claim 1 wherein the plug comprises material containing barium.

9. The percutaneous pedicle plug of claim 1 wherein the plug includes a titanium bead.

10. A method of inserting a percutaneous pedicle plug within a vertebral body, comprising:
a. creating a channel opening in the vertebral body;
b. forming threads within the channel opening of the vertebral body; and
c. inserting the percutaneous pedicle plug within the threads in the channel of the vertebral body.

11. The method of claim 10 wherein the channel in the vertebral body is created by means of a Jam-Sheady needle.

12. The method of claim 10 wherein the percutaneous pedicle plug is a Type-1 percutaneous pedicle plug.

13. The method of claim 10 wherein the percutaneous pedicle plug is a Type-2 percutaneous pedicle plug.

14. The method of claim 10 wherein the steps of creating the channel opening comprise:
a. forming the channel by a Jam-Sheady needle;
b. inserting a guide wire within the channel; and
c. removing the Jam Sheady needle.

15. The method of claim 10 wherein the steps of forming the threads in the channel comprise threading a threaded probe through the channel.

16. A method of inserting a percutaneous pedicle plug within a vertebral body, comprising:
a. forming a channel opening in the vertebral body by means of a Jam-Sheady needle wherein the Jam-Sheady needle includes a needle channel extending therethrough;
b. inserting a guide wire within the needle channel of the Jam-Sheady needle and the channel of the vertebral body;
c. removing the Jam Sheady needle;
d. inserting a threaded probe into the vertebral body channel wherein the threaded probe includes a channel for the guide wire;
e. forming threads within the channel opening of the vertebral body by means of the threaded probe;
f. removing the threaded probe and the guide wire from the vertebral body; and
g. inserting the percutaneous pedicle plug within the threads in the channel of the vertebral body.

17. The method of claim 16 wherein the percutaneous pedicle plug is a Type-1 percutaneous pedicle plug.

18. The method of claim 16 wherein the percutaneous pedicle plug is a Type-2 percutaneous pedicle plug.

19. A method of installing a percutaneous screw in a vertebral body comprising:
a. forming a channel opening in the vertebral body;
b. forming threads within the channel opening of the vertebral body by means of the threaded probe;
c. removing the threaded probe from the vertebral body;
d. inserting a percutaneous pedicle plug within the threads in the channel of the vertebral body, wherein the percutaneous pedicle plug includes a first end, a tapered second or tail end, and a threaded body to allow for threaded insertion of the percutaneous pedicle plug into the channel of the vertebral body, and a longitudinal central channel extending from the first end to the second end;
e. after a designated period of time removing the percutaneous pedicle plug from the vertebral body; and
f. installing the percutaneous screw within the channel in the vertebral body.

20. The method of claim 19 wherein the percutaneous pedicle plug is selected from the group consisting of a type-1 percutaneous pedicle plug or a type-2 percutaneous pedicle plug.
